(19) United States
(12) Patent Application Publication
(10) Pub. No.: US 2024/0358878 A1
(43) Pub. Date: Oct. 31, 2024

(54) Diffusing Device

(71) Applicant: Arome D'Art, Newport Beach, CA (US)

(72) Inventors: Douglas Patton, Newport Beach, CA (US); Jeffrey Thompson, Newport Beach, CA (US)

(73) Assignee: Arome D'Art, Newport Beach, CA (US)

(21) Appl. No.: 18/767,845

(22) Filed: Jul. 9, 2024

Related U.S. Application Data
(63) Continuation-in-part of application No. 17/055,965, filed on Nov. 16, 2020, now Pat. No. 12,029,837.
(60) Provisional application No. 62/743,497, filed on Oct. 9, 2018.
(60) Provisional application No. 62/632,715, filed on Feb. 20, 2018.

(51) Int. Cl. A61L 9/12

(52) U.S. Cl. CPC A61L 9/122; A61L 2209/12; A61L 2209/133

(57) ABSTRACT

Various embodiments of diffusing devices are described. The diffusing device can include a cover or retaining ring that attaches to the diffusing device to hold a scent cartridge in place. The scent cartridge can include a top plate suspended above the cartridge and preferably supported solely by the cartridge such that air flowing from the scent cartridge is circumferentially dispersed from the scent cartridge.

This application claims priority to U.S. non-provisional application having Ser. No. 17/055,965 filed on Nov. 16, 2020 (now U.S. Pat. No. 12,029,837), which is a § 371 national phase application of PCT application no. PCT/US19/18827 filed on Feb. 20, 2019, which itself claims priority to U.S. provisional application having Ser. No. 62/632,715 filed on Feb. 20, 2018 and U.S. provisional application having Ser. No. 62/743,497 filed on Oct. 9, 2018. These and all other referenced extrinsic materials are incorporated herein by reference in their entirety. Where a definition or use of a term in a reference that is incorporated by reference is inconsistent or contrary to the definition of that term provided herein, the definition of that term provided herein is deemed to be controlling.

### FIELD OF THE INVENTION

The field of the invention is diffusing devices.

### BACKGROUND

The following description includes information that may be useful in understanding the present invention. It is not an admission that any of the information provided herein is prior art or relevant to the presently claimed invention, or that any publication specifically or implicitly referenced is prior art.

In general, fragrance diffusing devices include a base unit and a cartridge that attaches to the base unit. A fragrance often in liquid form is then wicked or otherwise pulled from the cartridge, such that the fragrance passes through the base unit before exiting the device. Such devices are problematic as they retain previous scent(s) even when a different cartridge is used, they fail to diffuse the scent circumferential from the device, and are often eyesores that people attempt to hide by placing them in locations where such fragrance takes longer to reach the preferred location.

All publications identified herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Thus, there is still a need for improved diffusing devices and cartridges that address the above-identified problems.

### SUMMARY OF THE INVENTION

The inventive subject matter provides apparatus, systems and methods for dispensing units preferably made to receive a scent cartridge that can be used to disperse a fragrance from the scent cartridge to an area surrounding the dispensing unit.

Contemplated dispensing units preferably comprise a base with lower and upper surfaces, wherein the base may include a fan and a power source configured to power the fan. Contemplated power sources include, for example, one or more batteries and an input to receive a line voltage or other power source.

The base can be configured to receive a scent cartridge which can be inserted at least partially or fully within the base. It is preferred that the scent cartridge comprises a top plate suspended above an upper surface of the scent cartridge by a post that extends upwardly away from the scent cartridge to the top plate. In such embodiments, it is contemplated that the top plate may be solely supported by the post and therefore by the scent cartridge. In addition to helping to cover the scent cartridge from view, the top plate preferably causes air flowing from the scent cartridge to be circumferentially or otherwise evenly dispersed from the scent cartridge.

The top plate advantageously can include an artwork or kinetic arrangement while also hiding the scent depository beneath the top plate.

In some embodiments, the base may further comprise a first set of magnets that can be disposed at or beneath the upper surface of the base.

The diffusing unit can further include a cover having lower and upper surfaces, and that is configured to be coupled with the base to secure the scent cartridge between the cover and the base.

In some embodiments, the lower surface of the cover may further comprise a second set of magnets having a polarity that is opposite of the first set of magnets such that the cover is magnetically attached to the base via a magnetic coupling between the first and second sets of magnets.

In such embodiments, it is contemplated that the base or cover can include a third set of magnets. Where the third set of magnets is disposed at or beneath the upper surface of the base, the third set of magnets have a polarity that is the same as that of the second set of magnets. Where the cover comprises the third set of magnets, the third set of magnets have a polarity that is the same as that of the first set of magnets. In either case, by rotating the cover with respect to the base, the magnetic coupling changes to a repulsive force assisting in removing the cover from the base.

It is contemplated that the base can include a recessed central portion surrounded by an exterior wall that forms the upper surface of the base, where the recessed central portion is configured to receive a cartridge. In such embodiments, the scent cartridge can be disposed on the recessed portion of the base, and thereby be disposed adjacent to and above the fan in the base.

Contemplated scent cartridge can include a fragrance depository where the scent or fragrance is stored before being dispersed. In preferred embodiments, the fragrance depository comprises a plurality of scented beads that evaporate over time depending upon airflow. However, the fragrance depository could alternatively comprise a gel or liquid that is stored within the housing. Contemplated scented beads include ethylene-vinyl acetate (EVA) beads that have previously absorbed one or more scented solutions.

Advantageously, because scented air flows from the fan to the scent depository of the scent cartridge and then the scented air exits the scent cartridge, the scented air does not flow through the base. And because the scent cartridge with the top plate can be removed in whole, when the scent cartridge is replaced and a new scent cartridge is inserted, there is unlikely to be any remaining portion of the prior scent on the base. Thus, the unit does not send essential oils or other fragrance through the diffuser itself before it is dispersed into the outside air from the scent cartridge. This ensures a pure scent that does not mix with the internal electronics or housing of the unit. In addition, this can reduce or eliminate the buildup of bacteria residue in prior art devices that are difficult to clean and often must be thrown away with the scent cartridge or over time.

Various objects, features, aspects, and advantages of the inventive subject matter will become more apparent from the following detailed description of preferred embodiments, along with the accompanying drawing figures in which like numerals represent like components.

### BRIEF DESCRIPTION OF THE DRAWINGS

FIG. 1 is a perspective view of one embodiment of the device.

FIG. 2 is a vertical, cross-section view of the device of FIG. 1.

FIG. 3 is another perspective view of the device of FIG. 1.

FIGS. 4-6 are various exploded views of the device of FIG. 1.

FIG. 7 is a top view of one embodiment of a base unit.

FIG. 8 is a bottom view of the base unit of FIG. 7.

FIG. 9 is an enlarged, cross-section view of the device of FIG. 1.

FIG. 10 is an exploded view of one embodiment of a scent cartridge.

FIGS. 11-13 are top views of various embodiments of top plates for a scent cartridge.

FIGS. 14A-14B are top views and an enlarged, cross-section view, respectively, illustrating flow of scented air within and from the cartridge.

FIG. 15 is a table illustrating exemplary schedules of time that can be selected to determine how long the unit should be powered on and then off.

FIG. 16 is a perspective view of another embodiment of the device.

FIGS. 17A-17B are top perspective and bottom perspective views, respectively, of another embodiment of a cartridge.

FIG. 17C is a side view of the cartridge of FIGS. 17A-17B.

### DETAILED DESCRIPTION

Throughout the following discussion, numerous references will be made regarding servers, services, interfaces, portals, platforms, or other systems formed from computing devices. It should be appreciated that the use of such terms is deemed to represent one or more computing devices having at least one processor configured to execute software instructions stored on a computer readable tangible, non-transitory medium. For example, a server can include one or more computers operating as a web server, database server, or other type of computer server in a manner to fulfill described roles, responsibilities, or functions.

The following discussion provides many example embodiments of the inventive subject matter. Although each embodiment represents a single combination of inventive elements, the inventive subject matter is considered to include all possible combinations of the disclosed elements. Thus, if one embodiment comprises elements A, B, and C, and a second embodiment comprises elements B and D, then the inventive subject matter is also considered to include other remaining combinations of A, B, C, or D, even if not explicitly disclosed.

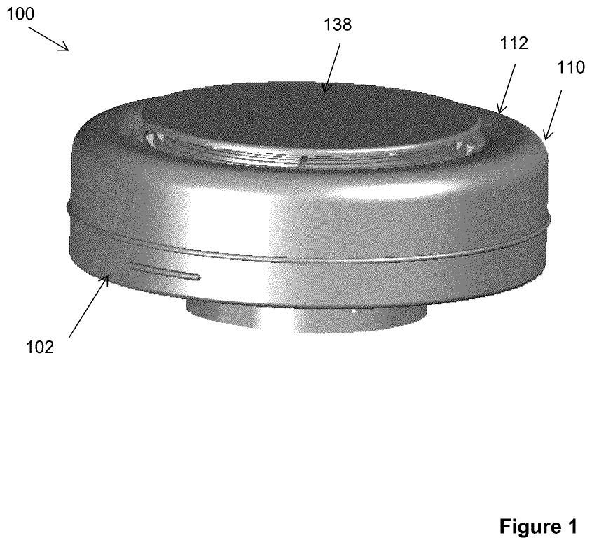

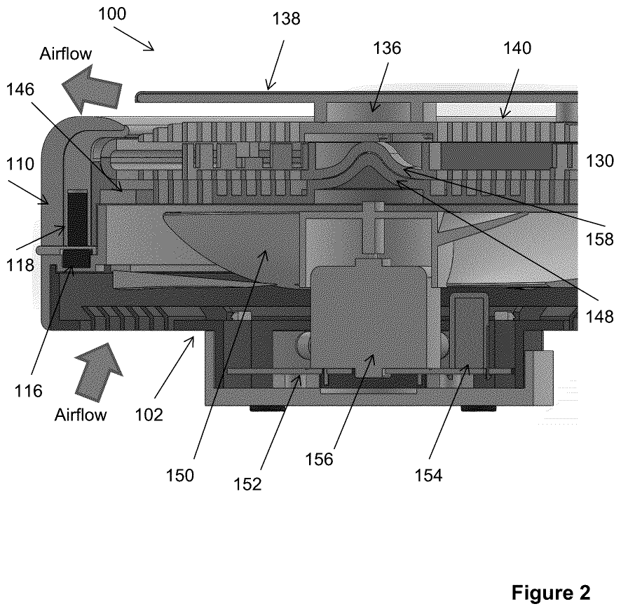

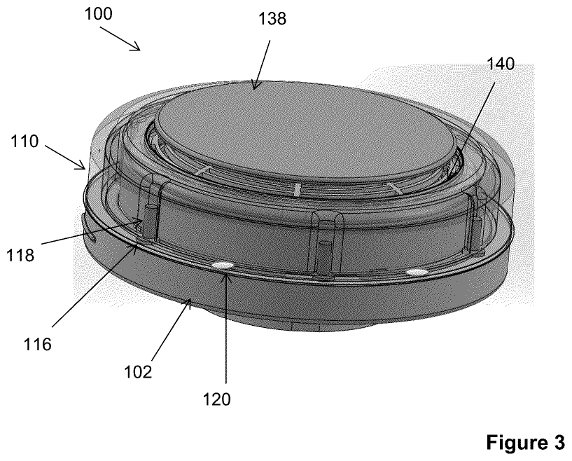

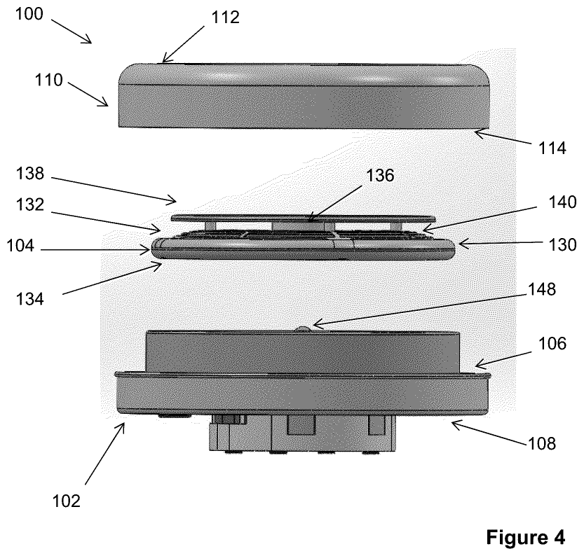

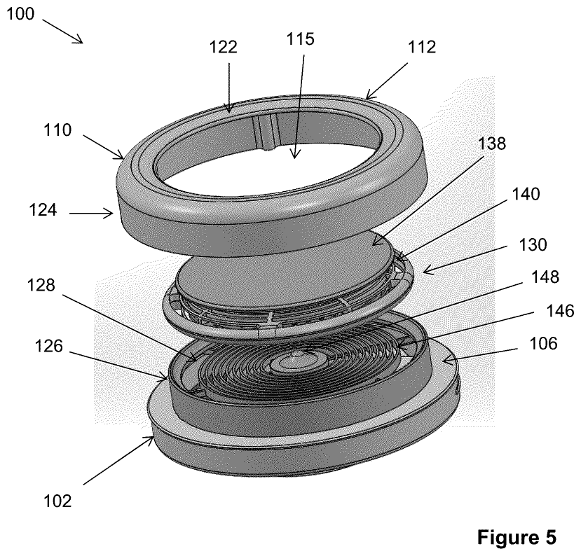

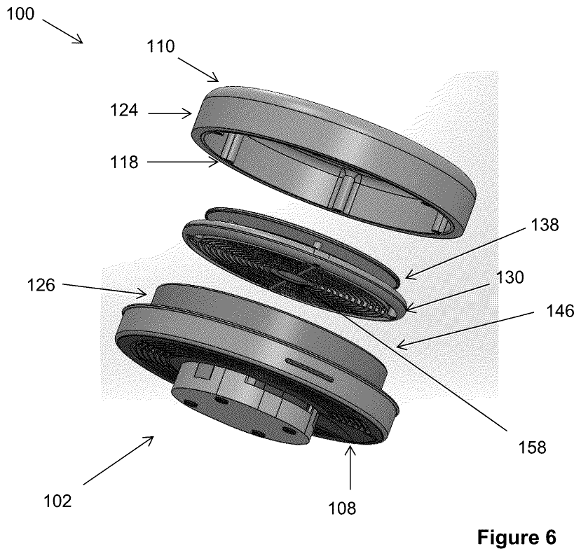

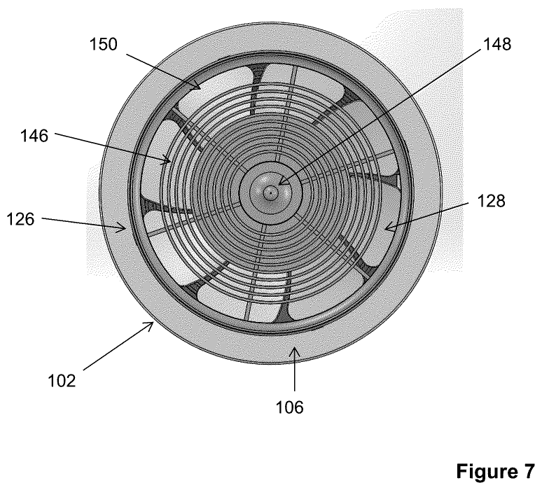

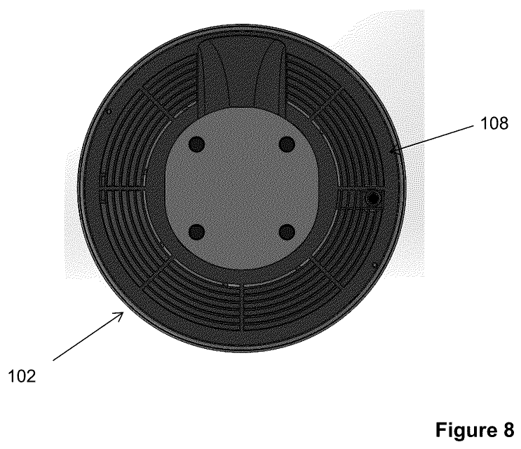

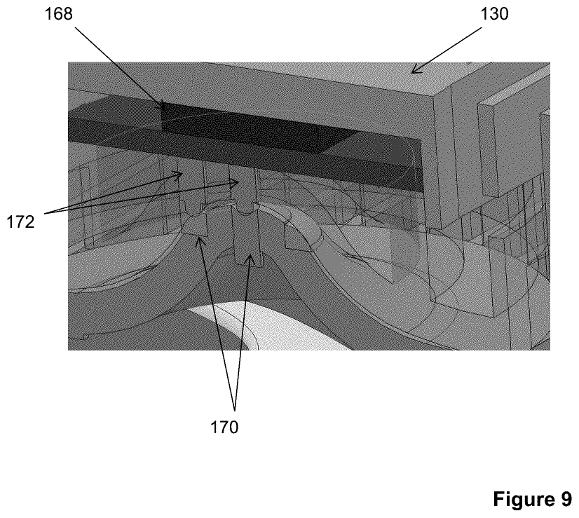

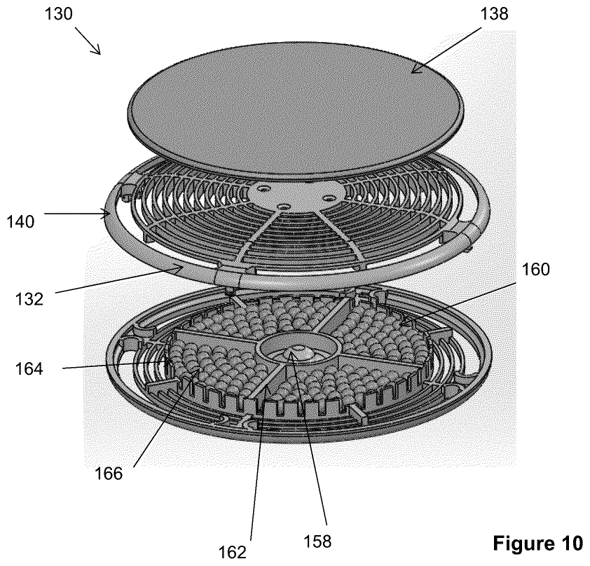

FIGS. 1-2 illustrates one embodiment of a fragrance dispensing unit 100 having a scent cartridge 130 disposed within the unit 100. The unit 100 comprises a base 102 and a cover or ring 110 is magnetically or otherwise coupled to the base 102. When the cover 110 is attached to the base 102, the scent cartridge 130 can be secured between the cover 110 and the base 102 to collectively maintain the scent cartridge 130 within the unit 100.

The base 102 comprises an upper surface 106 and a lower surface 108. Within the base 102 is disposed a fan 150, which is connected to a motor 156 that drives the fan 150. The base can also include electronics 152 that control the motor 156 and thereby can vary the fan speed or turn the fan 150 on or off. A battery 154 can be included for power storage and to power the various components of the base 102. Alternatively, or additionally, the base 102 can be coupled to a line voltage to charge the battery 154 and/or power the unit 100. In some embodiments, the battery 154 can be charged via induction.

The base 102 can include a grill 146 disposed above the fan 150, and that includes a plurality of apertures through which air can flow from the fan and through the grill 146. In this manner, air can flow through the lower surface 108 or other opening of the base 102 as shown by arrow, which then flows past the fan 150 and through the grill 146.

A first set of magnets 116 can be disposed at or beneath the upper surface 106 of the base 102, such that each of the magnets has the same polarity in the portion of the magnet nearest the upper surface 106. Thus, for example, where each magnet has a north and south pole end, all of the magnets 116 of the first set will have the same end facing toward the upper surface 106.

A second set of magnets 118 can be disposed at or above the lower surface 114 of the cover 110, such that each of the magnets has the same polarity in the portion of the magnet nearest the lower surface 114. It is especially preferred that the polarity of the second set of magnets 118 is opposite the polarity of the first set of magnets 116. In this manner, the magnetic attraction between the first and second sets of magnets 116, 118 can couple the cover 110 to the base 102.

In some contemplated embodiments, the base 102 can comprise a third set of magnets 120 disposed at or beneath the upper surface 106 of the base 102. In such embodiments, the third set of magnets 120 each has the same polarity in the portion of the magnet nearest the upper surface 106 and that polarity is opposite of the first set of magnets 116 and the same as the second set of magnets 118. In this manner, when the cover 110 is coupled with the base 102 and rotated with respect to the base 102, the third set of magnets 120 and the second set of magnets 118 will create a repulsive force due to the same polarity of the magnets, which will facilitate removal of the cover 110 from the base 102.

In other contemplated embodiments, the cover 110 can comprise a third set of magnets 120 disposed at or above the lower surface 114 of the cover 110. In such embodiments, the third set of magnets 120 each has the same polarity in the portion of the magnet nearest the lower surface 114 and that polarity is opposite of the second set of magnets 118 and the same as the first set of magnets 116. In this manner, when the cover 110 is coupled with the base 102 and rotated with respect to the base 102, the third set of magnets 120 and the first set of magnets 116 will create a repulsive force due to the same polarity of the magnets, which will facilitate removal of the cover 110 from the base 102.

When the cover 110 is coupled with the base 102, a lower surface 114 of the cover 110 abuts or rests on the upper surface 106 of the base 102. The cover 110 can also include a rim 122 that defines an upper surface 112 of the cover 110. The rim extends inwardly from a wall 124 of the cover 110.

As discussed above, the unit 100 can be configured to receive a scent cartridge 130 that is preferably disposable and removable from the base 102. The scent cartridge 130 has an upper surface 132 and a lower surface 134, with the upper and lower surfaces 132, 134 each having one or more apertures to permit air to flow through the cartridge from the fan 150, for example.

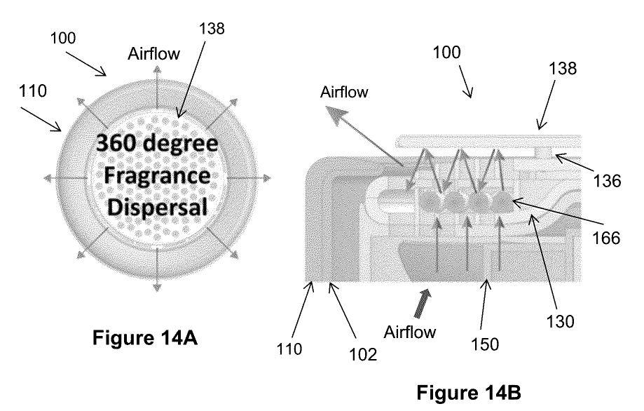

The scent cartridge 130 can include a post 136 extending upwardly from the upper surface 132, and that supports a top plate 138 that is suspended above the scent cartridge 130. In this manner, a first end of the post 136 can be attached to the scent cartridge 130, and the second opposing end of the post 136 can be attached to the top plate 138. The top plate 138 preferably is sized and dimensioned to cover or obscure all or a portion of the cartridge 130. Advantageously, the top plate 138 can cause some or all of the airflow to be directed circumferentially from the base 102 and cartridge 130, rather than simply directed upwardly as in many prior art devices. This helps to ensure an even scent distribution in a room, for example, from the cartridge 130. It is especially preferred that the cartridge 130 and top plate 138 collectively form a fragrance reverberation chamber 180 to maximize air flow past the fragrance portion (e.g., scented beads 166) of the cartridge 130 and then the top plate 138 causes the scented air to be evenly distributed from the cartridge 130 in a 360-degree circumference. This allows the unit 100 to quickly fill a room or other area with scented air. An example of a fragrance reverberation chamber 180 is shown in FIGS. 14A-14B.

The cartridge 130 can further include a grill 140 disposed above a scent repository 160 in the cartridge 130. The grill 140 ensures the scent repository 160 is not disturbed or that the beads 166 or other scented pieces are not removed from the cartridge 130. As shown, the scent repository 160 preferably comprises a one or more quadrants 164 (here, four quadrants), which each contains scented beads 166. Contemplated scented beads 166 include those formed from ethylene-vinyl acetate. However, in other embodiments, a scented gel or liquid could be disposed within the scent depository 160. The quadrants 164 can be formed by one or more walls 162 within the scent depository 160.

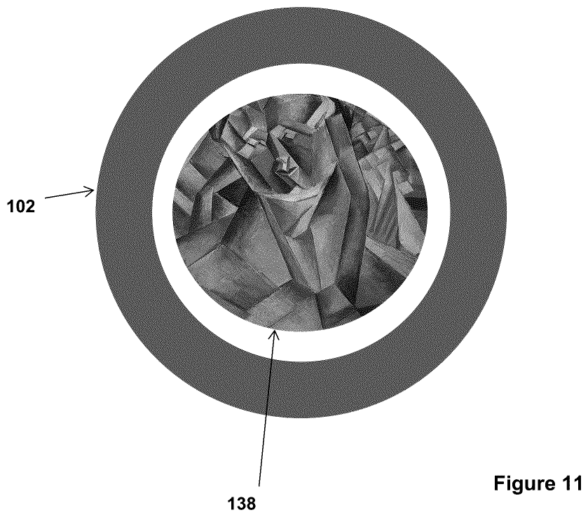

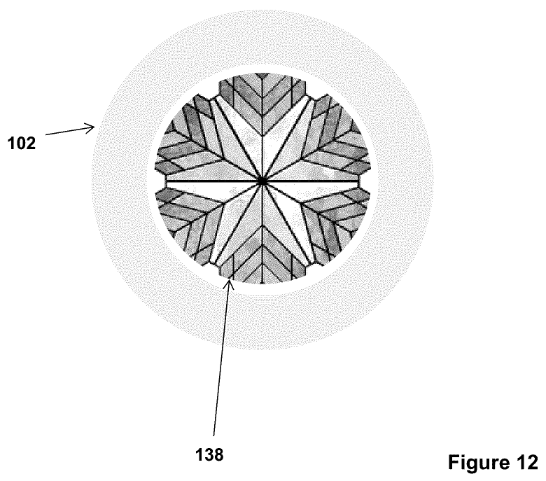

As shown in FIGS. 11-12, the top plate 138 can comprise an artwork which could include an image or a transparent or translucent portion(s) such as the stained glass replica shown in FIG. 12.

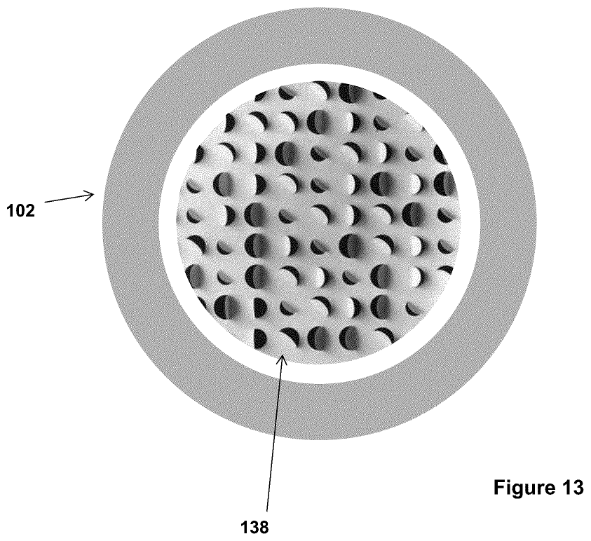

Alternatively, as shown in FIG. 13, the top plate 138 can comprise a kinetic arrangement or sculpture, which itself can change in visual appearance based on airflow passing through the kinetic arrangement or contacting a lower surface thereof. For example, the top plate can include a pressed-paper portion that can animate/flutter when the fan is in use.

The scent cartridge 130 preferably rests within a recessed area 128 of the base 102 formed at least partially by wall 126. The recessed area 128 can include a dimple or other projection 148. When the cartridge is positioned within the recessed area 128, the projection 148 is inserted into a recess 158 on the cartridge 130. This helps ensure proper placement of the cartridge 130 within the recessed area 128. The recessed area 128 is disposed above the fan 150.

Once the cartridge 130 is inserted within the recessed area 128 of base 102, the cover or ring 110 can be placed on an upper surface 132 of the cartridge 130, such that rim 122 of the cover 110 rests on the upper surface 132 of the cartridge and keeps the cartridge 130 within the recessed area 128. In such embodiments, it is contemplated that an upper surface 132 of the scent cartridge 130 is flush with an upper surface of the wall 126. Thus, a diameter of the rim 122 is less than a diameter of the cartridge 130.

The upper surface 112 of the cover 110 can include an aperture 115 defined by the rim 122. In this manner, when the cover 110 is attached to the base 102, the post of the cartridge 130 extends through the aperture 115 and the top plate 138 will extend above the cover 110.

When the fan 150 is turned on, air enters through a lower surface 108 of the base 102, past the fan 150, through the grill 146 of the base 102. From there, the non-scented air enters the scent cartridge 130 through its lower surface 134, passes through the scent depository 160, and exists through grill 140 of the cartridge 130. The now scented air exits through the space between the grill 140 and the top plate 138. As discussed above, the top plate 138 advantageously can cause the scented air to disperse circumferentially from the cartridge 130. In this manner, scented air flows from the scent cartridge 130 and out of the device 100 without passing through the base unit 102. Thus, little to no residual fragrance will be left on base 102 when the cartridge 130 is removed from the base 102, as the change in fragrance cartridge 130 will change the grill 140 through which the scented air passes so that different scents are not mixed.

In some embodiments, the cartridge 130 can include one or more light sources. It is contemplated that the light sources can be configured to illuminate during specific times of day either through a timer, a direct on/off control, or a signal received by an ambient light sensor. Where the top plate comprises a transparent or translucent portion, the light source can add to the visual effect of the top plate's artwork or kinetic arrangement.

It is contemplated that the electronics 152 can be configured to monitor a lifespan of the cartridge 130. Preferably, both the cartridge 130 and the base unit 102 have electrical contacts that contact one another when the cartridge 130 is placed on to the base unit 102, such as described above. In one embodiment, the dimple or other projection 148 could include an electrical contact 170 that contacts an electrical contact 172 disposed in recess 158 of the cartridge 130.

For example, when a cartridge 130 is first inserted into the base unit 102, the electronics can begin a timer that indicates how long the cartridge 130 has been inserted into the base unit 102. Via the electrical connection between the cartridge 130 and the base unit 102, the electronics could receive information about the cartridge, such as the type of cartridge 130 and some code, indicia or other means to indicate that the cartridge 130 is authentic and not a knock off cartridge. The information could also include a serial number or other unique identifier to prevent reuse of the cartridge 130 and to ensure that if the same cartridge is removed and reinserted, the timer does not reset.

It is further contemplated that the electronics or circuitry can be used to control an output of the unit 100. It is preferred that the electronics or circuitry could be used to create a series of intermissions between sequential outputs of scented air from the cartridge. Thus, the fan 150 could be powered on for a set time period followed by a second period of time where the fan 150 is powered off. This can be repeated indefinitely or for a set time period, for example.

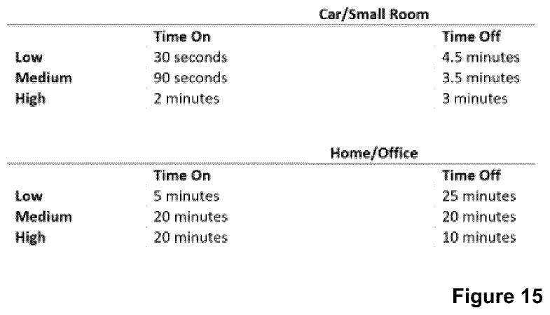

FIG. 15 illustrate a table providing examples of time periods where the fan is powered on and corresponding time periods where the fan is powered off. Thus, as one example, a user having the unit disposed in a car or a small room who prefers a low amount of fragrance may select the first option where the unit powers on the fan for 30 seconds followed by four and a half minutes where the fan is powered off. In some embodiments, the user could set the schedule to continue for a fixed number of hours or during certain times of the day and/or days of the week. It is contemplated that the schedule could be set via a separate application that is used to communicate with the unit.

The electronics 152 can comprise a processor, a memory with stored algorithms, and a PCB with other components. One of the stored algorithms can calculate a current lifespan of the cartridge 130 by calculating the amount of time the cartridge 130 has been open and inserted into the base 102, and the amount of time the fan 150 has been on while the cartridge 130 has been inserted within the base 102. Based on known evaporation rates of the scented beads 166 or other items in the scent depository 160, the electronics 152 can multiply the evaporation rate of the scented beads 166 by the time the cartridge has been inserted but the fan 150 off, as well as the evaporation rate of the scented beads 166 by the time the cartridge has been inserted and the fan 150 on. Adding these two values will allow the electronics 152 to determine the remaining percentage or amount of scent available in the cartridge 130.

Some of the information such as evaporation rates and total amount of scent of the cartridge 130 could be stored in the cartridge 130 and transferred to the base 102, transferred to the base from a remote source, or stored in the base 102. This is important as different cartridges may have different evaporation rates.

It is contemplated that the base 102 can include a transceiver on the electronics 152, for example, configured to transmit and receive information from a remote source such as a smart phone or other portable computing device. Thus, for example, the base 102 could transmit a remaining life of the cartridge to a user. In some embodiments, the user could control operation of the base 102 from the portable computing device. In other embodiments, the base 102 could transmit information to a user's device that can load a website, an image, or story that relates to the cartridge that was installed in the device 100.

In other contemplated embodiments, the cartridge 130 can include a memory and/or a microchip 168, which can be used to control fan speed in relation to scent aroma, for example. In addition, the microchip 168, or the electronics 152 of the base 102 can illuminate a light when the scent is expended. The electronics 152 and/or microchip 168 of the cartridge 130 can help to prevent the base 102 from working if a knockoff scent cartridge is being used.

It is further contemplated that the device 100 can be turned on/off by simply touching an annual ring or other portion of the base 102. This can allow powering on or off via capacitance. In addition, a bottom of the base 102 can have a slot to attach the base 102 to a mount or other support. In the slot can be a positive/negative connection so the mount or other support can transmit power to the base 102.

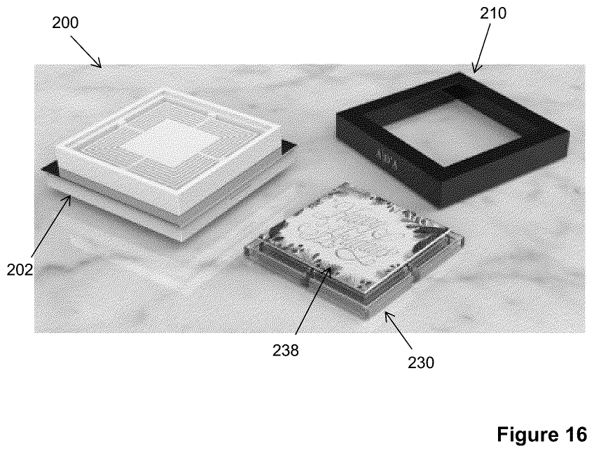

FIG. 16 illustrates another embodiment of fragrance dispensing unit 200 configured to receive a scent cartridge 230 disposed at least partially within a base 102 of the unit 100. The unit 200 is similar to the unit 100 described above, although the unit 200 comprises a square-shape rather than the circular shape of unit 100. Despite the difference in shape, the unit 200 functions the same as the unit 100 described above concerning airflow through the unit 200.

As shown, unit 200 preferably comprises a base 202 and a cover 210 coupled to the base 202. When the cover 210 is coupled to the base 202, the scent cartridge 230 can be secured between the cover 210 and the base 202 to collectively maintain the scent cartridge 230 within the unit 200.

As discussed above with respect to unit 100, the base 202 can comprise a fan which may be connected to a motor that drives the fan. Electronics may be used to control the motor and therefore control a speed of the fan (including powering the fan on and off).

The scent cartridge 230 preferably comprises a top plate 238, such as that described above, which can be supported by a post as discussed above. Advantageously, the top plate 138 can divert airflow from the cartridge out circumferentially from the base 202 and cartridge 230, rather than simply directed upwardly as in many prior art devices. This helps to ensure an even scent distribution in a room, for example, from the cartridge 230. It is especially preferred that the cartridge 230 and top plate 238 collectively form a fragrance reverberation chamber to maximize air flow past the fragrance portion of the cartridge 230 and then the top plate 238 causes the scented air to be evenly distributed from the cartridge 230 in a 360-degree circumference. This allows the unit 200 to quickly fill a room or other area with scented air.

As shown, the top plate 238 can comprise an artwork.

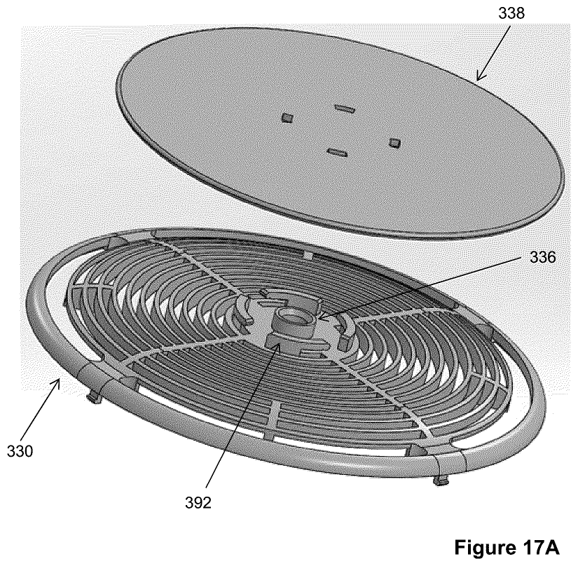

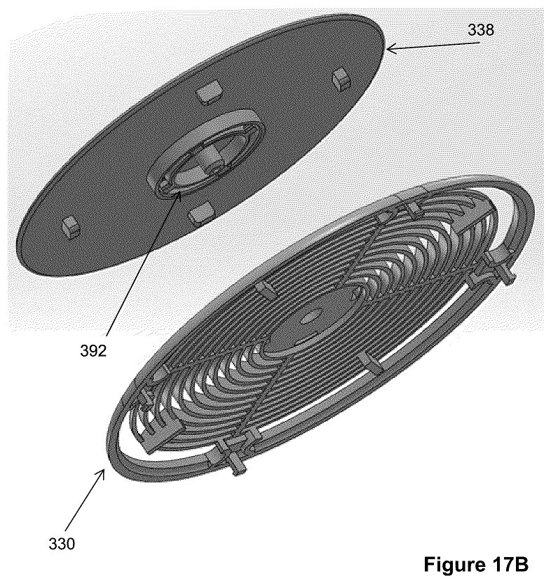

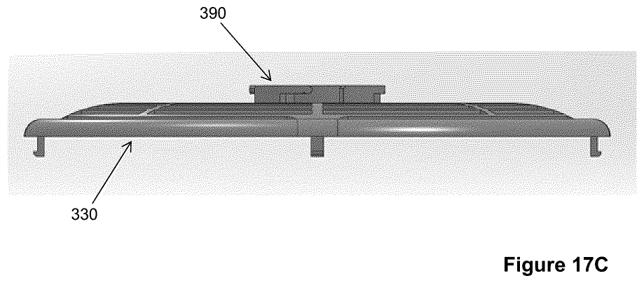

FIGS. 17A-17C illustrate another embodiment of a cartridge 330 having a removable top plate 338. The scent cartridge 330 can include a post 336 extending upwardly from an upper surface of the scent cartridge 330, and that supports the top plate 338 that is suspended above the scent cartridge 330. Thus, a first end of the post 336 can be attached to the scent cartridge 330, and the second opposing end of the post 336 can support the top plate 338. As discussed above, the top plate 338 can advantageously cause some or all of the airflow to be directed circumferentially from the cartridge 330, rather than simply directed upwardly as in many prior art devices.

In some embodiments, the top plate 338 can be secured to the cartridge 330 via one or more projections 390 that are configured to be inserted into corresponding receptacles 392 disposed on the top plate (best shown in FIG. 17B). Although four projections are shown, it is contemplated that the cartridge 300 could have more or fewer projections. Of course, it is also contemplated that the projections could instead be disposed on the top plate 338 and the receptacles disposed on the cartridge 330 without departing from the scope of invention.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

Unless the context dictates the contrary, all ranges set forth herein should be interpreted as being inclusive of their endpoints and open-ended ranges should be interpreted to include only commercially practical values. Similarly, all lists of values should be considered as inclusive of intermediate values unless the context indicates the contrary.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

The recitation of ranges of values herein is merely intended to serve as a shorthand method of referring individually to each separate value falling within the range. Unless otherwise indicated herein, each individual value with a range is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A fragrance dispensing unit, comprising:
a base having lower and upper surfaces;
a scent cartridge configured to be inserted within the base, wherein the scent cartridge comprises a top plate suspended above the upper surface of the scent cartridge by a post that extends upwardly away from the scent cartridge to the top plate, and wherein the top plate causes air flowing from the scent cartridge to be circumferentially dispersed from the scent cartridge; and
a cover configured to couple with the base to secure the scent cartridge between the cover and the base.

2. The fragrance dispensing unit of claim 1, wherein the base further comprises a fan and power source configured to power the fan.

3. The fragrance dispensing unit of claim 1, wherein the base comprises a recessed central portion surrounded by an exterior wall that forms the upper surface of the base, and wherein the recessed central portion is disposed above the fan, and wherein the scent cartridge is configured to be inserted within the recessed central portion of the base.

4. The fragrance dispensing unit of claim 3, wherein the recessed central portion comprises a projection or dimple that extends upwardly from the base, and wherein the scent cartridge is further configured to be inserted within the recessed central portion of the base, and wherein the scent cartridge comprises a recessed area configured to receive the projection or dimple.

5. The fragrance dispensing unit of claim 1,
a first set of magnets disposed at or beneath the upper surface of the base; and
wherein the cover comprises lower and upper surfaces, wherein the lower surface comprises a second set of magnets having a polarity that is opposite of the first set of magnets such that the cover is magnetically attached to the base.

6. The fragrance dispensing unit of claim 5, further comprising:
a third set of magnets disposed either (i) at or beneath the upper surface of the base and having a polarity that is the same as that of the second set of magnets, or (ii) at or above the lower surface of the cover and having a polarity that is the same as that of the first set of magnets, such that rotation of the cover with respect to the base when the cover is attached to the base causes the cover to be repelled from the base due to the same polarity of the third set of magnets with either the first or second set of magnets.

7. The fragrance dispensing unit of claim 6, wherein the third set of magnets are disposed at or beneath the upper surface of the base, and wherein the third set of magnets have a polarity that is the same as that of the second set of magnets, such that rotation of the cover with respect to the base when the cover is magnetically attached to the base causes the cover to be repelled from the base due to the polarity of the second and third set of magnets being the same.

8. The fragrance dispensing unit of claim 6, wherein the third set of magnets are disposed at or above the lower surface of the cover, and wherein the third set of magnets have a polarity that is the same as that of the first set of magnets, such that rotation of the cover with respect to the base when the cover is magnetically attached to the base causes the cover to be repelled from the base due to the polarity of the first and third set of magnets being the same.

9. The fragrance dispensing unit of claim 1, wherein the cover comprises an aperture on the upper surface, and a rim that extends inwardly from an outer surface of the cover.

10. The fragrance dispensing unit of claim 1, wherein the scent cartridge is further configured to be flush with the upper surface of the base when the scent cartridge is inserted within the base.

11. The fragrance dispending unit of claim 1, wherein the cover abuts the upper surfaces of the scent cartridge and base when the cover is coupled with the base.

12. The fragrance dispensing unit of claim 1, wherein the scent cartridge comprises a plurality of quadrants divided by one or more walls, and wherein each quadrant comprises a scent depository, and wherein each scent depository comprises a set of fragrance beads.

13. The fragrance dispensing unit of claim 1, wherein the top plate is sized and dimensioned to cover the cartridge.

14. The fragrance dispensing unit of claim 1, wherein the top plate comprises an artwork.

15. The fragrance dispensing unit of claim 1, wherein the top plate comprises a kinetic arrangement such that air flowing through or against the top plate causes movement of the kinetic arrangement and generates a visual change of the look of the top plate.

16. The fragrance dispensing unit of claim 1, wherein the scent cartridge comprises a light source, and wherein the light source causes a visual change in a kinetic arrangement or artwork disposed on the top plate.

17. The fragrance dispensing unit of claim 2, wherein the fan is configured to cause airflow through the scent cartridge such that scented air flows from the scent cartridge without passing through the base unit.

18. The fragrance dispensing unit of claim 1, wherein the scent cartridge comprises an electrical interface that contacts an electrical interface of the base when the scent cartridge is installed within the base.

19. The fragrance dispensing unit of claim 1, wherein the cover comprises an aperture on the upper surface, and a rim that extends inwardly from an outer surface of the cover, and wherein the top plate of the cartridge extends above the aperture, and wherein the rim abuts an upper surface of the cartridge.
